(19) United States
(12) Patent Application Publication
(10) Pub. No.: US 2015/0223954 A1
(43) Pub. Date: Aug. 13, 2015

(54) WIRE STENT

(71) Applicant: Industry-Academic Cooperation Foundation, Yonsei University, Seoul (KR)

(72) Inventors: Yang Soo Jang, Seoul (KR); Myeong Ki Hong, Seoul (KR); Dong Hoon Choi, Seoul (KR); Young Guk Ko, Seoul (KR); Jung Sun Kim, Seoul (KR); Byeong Keuk Kim, Seoul (KR)

(21) Appl. No.: 14/423,468

(22) PCT Filed: Aug. 26, 2013
(86) PCT No.: PCT/KR2013/007618

§ 371 Date: Feb. 24, 2015

(30) Foreign Application Priority Data
Aug. 24, 2012 (KR) 10-2012-0093175
Aug. 28, 2012 (KR) 10-2012-0094322

(51) Int. Cl. A61F 2/89

(52) U.S. Cl. CPC A61F 2/89; A61F 2250/0067

(57) ABSTRACT

The present invention relates to a wire stent. The present invention provides a wire stent consisting of a plurality of unit wires. The surfaces of the portions of the wire stent in which the plurality of unit wires are interconnected contact each other in the axial direction, thus increasing the strength of the wire stent in the radial direction while maintaining the unique flexibility of the wire stent, thereby minimizing a recoil phenomenon and shortening phenomenon. Further, the wire stent of the present invention is configured such that the width of the strut of the stent increases toward the inner wall of a blood vessel to thus enable endothelial cellularization to be easily performed, and the stent is fixed at the inner wall of the blood vessel in a more firm manner to thus be more effective in treating angiostenosis.

### TECHNICAL FIELD

The present invention relates a wire stent, and more particularly, to a wire stent capable of allowing connection portions in a stent consisting of wires to come in surface contact with each other to reinforce a radial strength.

Also, the present invention relates to a wire stent having a structure in which endothelial cellularization is easily performed during a vascular regeneration process after the stent is mounted at a blood wall upon surgical implantation of the stent.

### BACKGROUND ART

Generally, balloon catheters and stents are medical apparatuses which are surgically implanted inside a vascular lumen or a blood vessel to expand the vascular lumen or the blood vessel when the vascular lumen in the human body is narrowed by various diseases developed in the human body, resulting in degraded innate functions of the vascular lumen, or when diseases such as poor blood circulation caused by the narrowed blood vessel occur.

Among such stents, a wire stent is manufactured by winding wires in a predetermined form of a frame to shape the wires and welding connection portions using laser beams. Korean Unexamined Patent Application Publication Nos. 10-2008-0044323 and 10-2011-0051849 disclose one example of such a wire stent.

In the case of conventional wire stents, however, connection portions between unit wires are formed so that the unit wires come in point contact with each other. When such wire stents have the connection portions formed to come in point contact with each other, the wire stents have an advantage in flexibility, but have a problem in that the unwelded portions do not come in close contact with a curbed shape of a blood vessel as the stent swells, which makes it easy to deform the wire stents. Also, the conventional wire stents have a problem in that a recoiling or shortening phenomenon in which the stents are shortened due to plastic deformation after swelling.

Meanwhile, referring to FIGS. 8a and 8b, a conventional wire stent is subjected to laser cutting to be manufactured in the form of a wire. In this case, since a metal material is wound in a predetermined form of a frame to be shaped, and subjected to laser cutting, the cross section of a finally manufactured strut 1 is in a rectangular shape (see FIG. 8a). Also, when the metal material is connected as a wire without subjecting the metal material to laser cutting, the cross section of the strut 1 is in a circular shape (see FIG. 8b). A vascular regeneration process proceeds in state in which the strut whose cross section is in such a rectangular or circular shape is attached to a blood wall. That is, the endothelial cellularization occurs in both directions of the strut (in a direction of arrows or a direction opposite to the direction of arrows as shown in the drawing). However, when the cross section of the strut is in a rectangular or circular shape, endothelial cells do not easily climb over the strut, which makes it impossible to normally realize the endothelial cellularization.

### DISCLOSURE

### Technical Problem

Therefore, the present invention is designed to solve the problems of the prior art, and therefore it is an object of the present invention to provide a wire stent capable of enhancing the strength of the wire stent in a radial direction while maintaining the unique flexibility of the wire stent intact.

Also, it is another object of the present invention to provide a wire stent capable of promoting endothelial cellularization by designing both cross-sectional structures of the wire stent so as to promote the endothelial cellularization during a vascular regeneration process after the wire stent is mounted on a blood wall upon surgical implantation of the wire stent.

### Technical Solution

To solve the above problems, according to an aspect of the present invention, there is provided a wire stent comprising a plurality of unit wires to treat angiostenosis. Here, connection portions of the plurality of unit wires come in surface contact with each other in an axial direction.

According to one exemplary embodiment of the present invention, the plurality of unit wires may be connected to each other using at least one method selected from the group consisting of welding, and adhesion.

According to another exemplary embodiment of the present invention, the unit wires having a predetermined pattern may be configured to be connected with each other.

According to still another exemplary embodiment of the present invention, each of the unit wires may include a cell wire configured to form a cell with another adjacent unit wire; and a connection unit extending from one end of the cell wire to be connected with another unit wire.

According to still another exemplary embodiment of the present invention, the cell wire may have a zigzag shape in an axial direction.

According to still another exemplary embodiment of the present invention, connection portions of the connection units come in surface contact with each other.

According to still another exemplary embodiment of the present invention, the connection units may extend in opposite directions from both ends of the cell wire.

According to still another exemplary embodiment of the present invention, the stent may be made of at least one selected from the group consisting of a metal, a biodegradable polymer, a metal coated with the biodegradable polymer, a mixture of the metal and the biodegradable polymer, and a combination thereof.

According to still another exemplary embodiment of the present invention, in struts constituting the stent, the struts extending in an axial direction may be formed of a biodegradable polymer, and the struts extending in a circumferential direction may be formed of a metal.

According to still another exemplary embodiment of the present invention, the plurality of unit wires may be combined to form closed cells.

According to still another exemplary embodiment of the present invention, each of the struts constituting the stent may be formed so that the width of the strut increases toward the inner wall of a blood vessel.

According to still another exemplary embodiment of the present invention, both lateral surfaces of each of the struts are formed so that a slope of the strut increases toward the center thereof.

According to still another exemplary embodiment of the present invention, both lateral portions of each of the struts may be formed so that the lateral portions of the strut are symmetric to each other.

According to still another exemplary embodiment of the present invention, a cross section of each of the struts may be in a trapezoidal shape.

According to still another exemplary embodiment of the present invention, the cross section of each of the struts may be in a semicircular shape.

According to still another exemplary embodiment of the present invention, the cross section of each of the struts may be in triangular shape.

According to still another exemplary embodiment of the present invention, each of the struts may have a height of 30 to 120 μm.

According to still another exemplary embodiment of the present invention, a drug-carrying groove configured to carry a drug may be formed at one surface of each of the struts.

According to yet another exemplary embodiment of the present invention, both lateral surface portions of each of the struts constituting the wire stent are formed so that the both lateral surface portions of the strut are inclined in a straight or curved line.

### Advantageous Effects

According to one exemplary embodiment of the present invention, the wire stent including the wires can be useful in enhancing the strength of the wire stent in a radial direction while maintaining the unique flexibility of the wire stent intact by connecting the wires by means of welding and/or adhesion so that the connection portions of the wires in the stent do not come in point contact but come in surface contact with each other, thereby minimizing the recoiling and shortening phenomena.

According to one exemplary embodiment of the present invention, the wire stent can also be useful in being more firmly fixed in the inner wall of a blood vessel and more effectively treating angiostenosis by forming both lateral portions of each of struts constituting the wire stent so that the width of each of the struts increases toward the inner wall of the blood vessel so as to promote endothelial cellularization in a state in which the wire stent is mounted at the inner wall of the blood vessel.

### DESCRIPTION OF DRAWINGS

FIG. 1 is a developed view showing a developed state of a wire stent according to one exemplary embodiment of the present invention.

FIG. 2 is an enlarged front view of a connection portion as shown in FIG. 1.

FIGS. 3a and 3b are enlarged cross-sectional views of the connection portion as shown in FIG. 1.

FIG. 4 is a schematic view showing one example of a closed cell stent.

FIGS. 5 and 6 are schematic views showing one example of an open cell stent.

FIG. 7 is a graph illustrating the results obtained by comparing the radial forces of the closed cell stent and the open cell stent.

FIGS. 8a and 8b are configuration views schematically showing the cross section of a strut in a conventional wire stent.

FIG. 9 is a configuration view schematically showing the cross section of a strut in the wire stent according to one exemplary embodiment of the present invention.

FIGS. 10 and 11 are configuration views schematically showing the cross sections of struts in a wire stent according to another exemplary embodiment of the present invention.

FIG. 12 is a graph comparing the results of a cell migration assay according to an angle formed between the wire strut and the inner wall of a blood vessel.

### BEST MODE

In the present invention, the “wire stent” may refer to a stent including a plurality of unit wires.

In the present invention, the term “wires” may refer to strands constituting the stent.

In the present invention, the term “struts” may refer to individual strands constituting the stent.

In the present invention, the term “cell” may refer to a void space formed by the wires.

In the present invention, the term “closed cell” may refer to a closed cell completely surrounded by the wires.

In the present invention, the term “open cell” may refer to a cell which is not completely surrounded by the wires but partially opened.

Hereinafter, the wire stent according to one exemplary embodiment of the present invention will be described in detail with reference to the accompanying drawings.

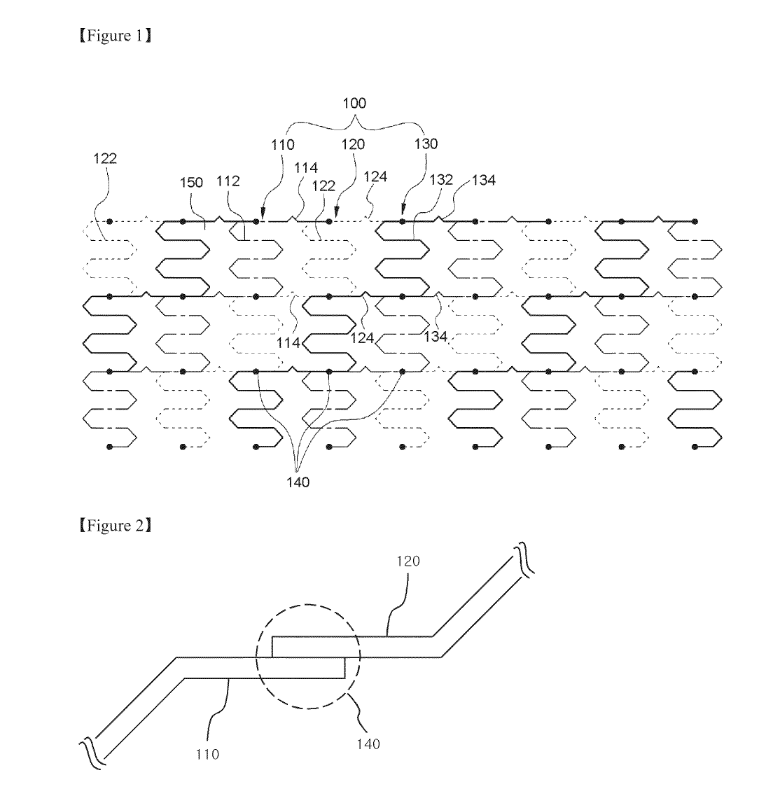

FIG. 1 is a developed view showing a developed state of a wire stent according to one exemplary embodiment of the present invention. For example, the wire stent may have an entire shape such as an elongated tubular shape. However, the shape of the wire stent is not limited to the tubular shape. For example, the wire stent may be formed in different shapes. Hereinafter, the wire stent having a tubular shape will be representatively described in detail.

The wire stent is inserted into a blood vessel, and closely attached to the inner wall of the blood vessel upon surgical implantation of the wire stent. The wire stent closely attached to the inner wall of the blood vessel serves to expand the blood vessel so as to promote blood circulation. The wire stent may be made of a material having predetermined rigidity and elasticity.

In the present invention, the wire stent may be made of a metal and/or a biodegradable polymer. Specifically, for example, the wire stent may be made of 1) a metal alone, 2) a biodegradable polymer alone, 3) a metal coated with the biodegradable polymer, 4) a mixture of the metal and the biodegradable polymer, or 5) a combination of two or more of the components 1) to 4).

When the metal and the biodegradable polymer are used together, the wire stent has an advantage in that it is possible to enhance radiopacity.

At least one selected from the group consisting of stainless steel, cobalt, titanium, platinum, nickel, iridium, niobium, tantalum, gold, silver, copper, aluminum, chromium, manganese, magnesium, and an alloy thereof may be selected and used as the metal.

By way of example, nitinol may be used as the alloy. In this case, nitinol is a kind of a Ni—Ti alloy and an alloy having shape memory behavior. The most common alloy is 55-nitinol which includes Ni at 54 to 56% by weight and the balance of Ti. The nitinol alloy has good corrosion resistance, no magnetism, and a relatively low density of 0.234 lb/in³.

The biodegradable polymer generally encompasses all types of polymers which are autonomously decomposed in a living body or a natural environment. In the present invention, for example, a copolymer or homopolymer of lactic acid and glycolic acid; a polymer including a carbohydrate-derived monomer such as a glucose derivative as a constituent element; a biodegradable hydrogel such as alginic acid; or a natural polymer such as a polypeptide, a polysaccharide, or a polynucleotide may be used as the biodegradable polymer. Examples of the biodegradable polymer may include polylactide (PLA), poly-L-lactide (PLLA), polyglicolide (PGA), polylactide-co-glicolide (PLGA), poly-ε-caprolactone (PCL), polylactide-co-caprolactone (PLCL), polydioxanone (PDO), poly-β-hydroxybutyrate (PHB), and the like, which may be used alone or in combination.

Meanwhile, in the struts constituting the wire stent, the struts extending in an axial direction (in a left/right direction as shown in FIG. 1, that is, a longitudinal/horizontal/transverse direction of the blood vessel and the tubular shape) may be formed of the biodegradable polymer, and the struts extending in a circumferential direction (in an up/down direction as shown in FIG. 1, that is, a radial/vertical/machine direction of the blood vessel and the tubular shape) may be formed of the metal. Here, the struts in the axial direction does not refer to struts having a linear shape parallel to the axis, and may encompass struts inclined at an angle of approximately ±40°, ±30°, ±20°, or ±10°, based on the axis. Also, the struts in the axial direction may have a curved section or a mixed straight/curved section. The struts in the circumferential direction are applicable in the same manner as in the struts in the axial direction.

Meanwhile, the wire stent may have a two-layer structure including an inner stent and an outer stent. In this case, a polymer fiber may be inserted between the inner stent and the outer stent. Here, the polymer may be a biodegradable polymer, and may be stacked in the form of a fiber sheet.

The connection between the wires may be performed using a welding and/or adhesion method. The welding may be performed using a typical welding method, and the adhesion may also be performed using a typical adhesive.

In the case of the adhesion, when the biodegradable polymer is used as a stent material, the polymer may be melted, and may be used for attachment. The same polymers as the biodegradable polymer constituting the wire stent may be used as the polymer used for adhesion, or the same or similar types of polymers may also be used as the polymer used for adhesion. When the polymer is melted, the polymer may be melted using a solvent, or melted using a method such as heating.

Also, the wires may also be connected using an adhesive. For example, bioadhesives such as a cyanoacrylate-based glue, a fibrin glue, and a protein gelatin glue may be used as the adhesive.

Referring to FIG. 1, the wire stent includes a plurality of unit wires 100 arranged in an axial direction. The plurality of, at least two, unit wires 100 may be connected with each other. In this exemplary embodiment, the connection of the three unit wires 100 is shown in FIG. 1. That is, a first unit wire 110, a second unit wire 120, and a third unit wire 130, all of which have a certain pattern, may be connected to each another.

In the configuration of the unit wires 100, the first unit wire 110 will be specifically described by way of example. The first unit wire 110 includes a first cell wire 112 configured to form a cell 150 with other unit wires 120 and 130, and connection units 114 extending from both ends of the first cell wire 112.

The first cell wire 112 may be used to form the entire backbone of the wire stent, and thus may have a zigzag shape in a circumferential direction, as specifically shown in FIG. 1. That is, the first cell wire 112 has a shape in which a plurality of valleys and ridges are repeatedly formed. Of course, the first cell wire 112 may have various shapes such as a net shape other than the shape as shown in FIG. 1. The first cell wire 112 forms a cell 150 having a compartmentalized space with adjacent second and third cell wires 122 and 132. The size and cross section of the cell 150 may be generally determined according to a degree of expansion required in consideration of the diameter of a blood vessel.

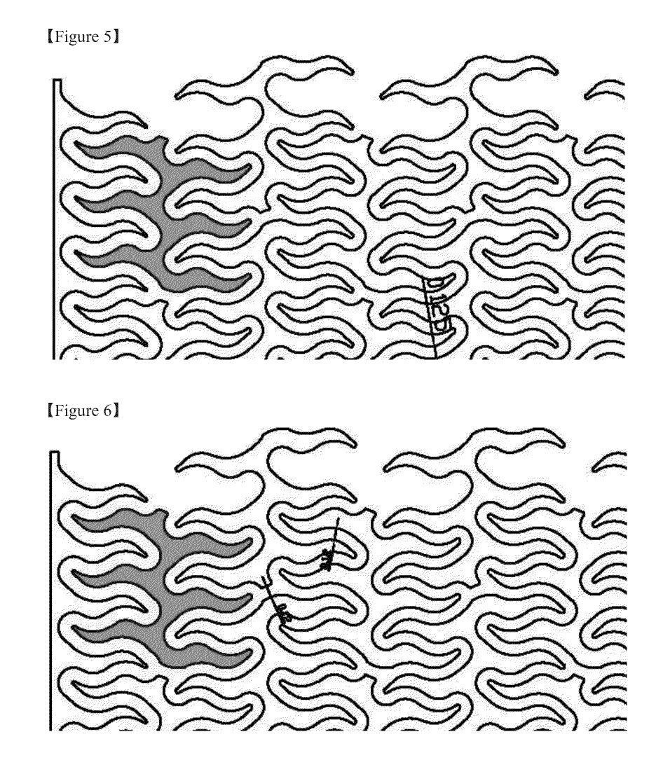

The cell 150 may be a closed cell, or an open cell. The closed cell may have a better radial force than the open cell, and thus the closed cell may be advantageous in terms of minimization of the recoiling and shortening phenomena. As shown in FIG. 1, the cell 150 is formed as the closed cell. In this case, the closed cells are sparsely formed at a slightly large area, as shown in FIG. 1. As shown in FIG. 4, however, the closed cells may be compactly formed at a reduced area, as necessary.

Also, the connection unit 114 extending from one end of the first cell wire 112 may be connected to other unit wires 120 and 130. More particularly, the connection units 114 may extend in opposite directions, that is, extend upward and downward from the first cell wire 112, based on the wire stent as shown in FIG. 1. The connection units 114 extending from the first cell wire 112 are connected to the adjacent second and third unit wires 120 and 130, respectively. Also, connection portions 140 of the connection units 114 connected to the second and third unit wires 120 and 130 come in surface contact with each other.

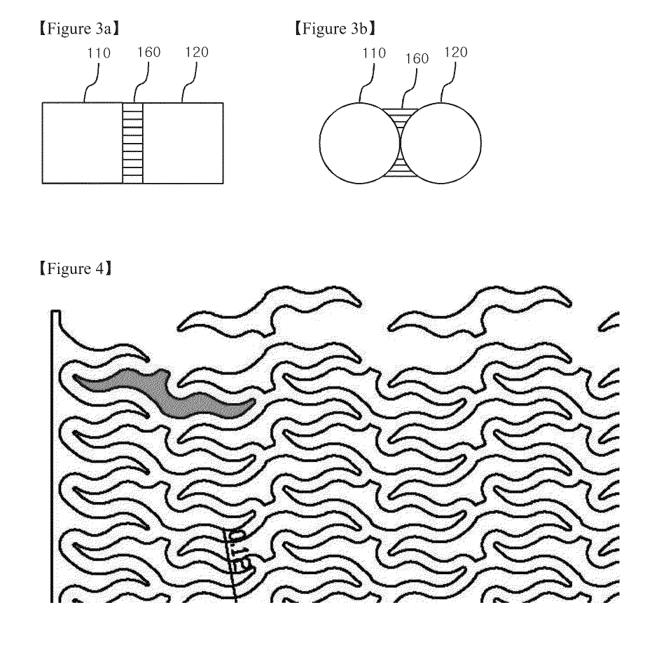

FIG. 2 is an enlarged front view of a connection portion as shown in FIG. 1, and FIG. 3 is an enlarged cross-sectional view of the connection portion as shown in FIG. 1.

As shown in FIG. 2, the first unit wire 110 and the second unit wire 120 form a surface contact at a connection portion 140 in an axial direction.

In the present invention, the connection between the unit wires 110, 120 and 130 whose connection portions come in surface contact with each other may include all connection between the cell wires 112, 122 and 132, connection between the connection units 114, 124 and 134, connection between the cell wires 112, 122 and 132 and the connection units 114, 124 and 134.

As shown in FIG. 2, when two wires are welded or joined, each strut in the connection portion may have the width of ½, as necessary, so that the width of the connection portion is identical to that of an unwelded portion even after each strut is welded. That is, the welding may be performed so that there is no change in the widths of the connection portion and a non-connection portion.

FIGS. 3a and 3b are diagrams showing the connection portion 140, as viewed from the cross section of a wire, FIG. 3a shows that the cross section of the wire is in a rectangular shape, and FIG. 3b shows that the cross section of the wire is in a circular shape. Of course, the cross section of the wire may have various shapes such as triangular, trapezoidal, semicircular shapes, in addition to the rectangular and circular shapes.

As shown in FIG. 3, the first unit wire 110 and the second unit wire 120 may be connected via a joint portion 160 while coming in surface contact with each other. The joint portion 160 may be formed using a welding and/or adhesion method. When the cross section of the wire is in a rectangular shape, it is easy to form a surface contact. However, when the cross section of the wire is in a circular shape, it is also possible to form the surface contact by means of welding.

In the plurality of unit wires 100 as described above, the connection portions 140 preferably come in surface contact in an axial direction. As the connection portions 140 come in surface contact, the strength of the wire stent may increase in a radial direction. Specifically, when the connection portions 140 come in point contact, the wire stent may be deformed when swelled since the wire stent does not match with the curved shape of a blood vessel.

As described in this exemplary embodiment, however, when the connection portions 140 of the unit wires 100 come in surface contact with each other, the strength of the wire stent increase in a radial direction (a vertical direction as shown in FIG. 1). As a result, the wire stent may be closely attached to the inner wall of the blood vessel while maintaining the rigidity of the wire stent in a state in which the wire stent matches with the curved shape of the blood vessel. Also, it is possible to prevent the recoiling and shortening phenomena of the conventional stents. On the other hand, the connection portions 140 may be connected using various methods. Generally, the connection portions 140 may be connected by means of welding and/or adhesion.

According to this exemplary embodiment, the connection units 114, 124, and 134 are provided at both ends of the unit wires 100, as described above. As the connection portions 140 come in surface contact, the connection units 114, 124, and 134 may serve to reinforce degraded flexibility of the wire stent.

That is, since the connection units 114, 124, and 134 connect the respective unit wires 100 at predetermined intervals in a circumferential direction of the wire stent, the wire stent may also secure flexibility while maintaining a predetermined strength in a radial direction. When the flexibility of the wire stent is secured as described above, the wire stent may be easily swelled to match with the shape of the blood vessel.

The connection portions 140 of the above-described unit wires 100 may be properly selected according to the material and thickness of the wire stent. That is, the rigidity of the wire stent may also be enhanced by lengthening the connection portions 140. Also, when the flexibility of the wire stent needs to be further enhanced, it is possible to design the connection portions 140 to be short.

Hereinafter, the performance of the closed cell stent and the open cell stent was tested for comparison. For this purpose, one closed cell stent (FIG. 4) and two open cell stents (FIGS. 5 and 6) designed as shown in FIGS. 4 to 6 and listed in Table 1 were manufactured. FIG. 4 is a schematic view showing one example of a closed cell stent, FIGS. 5 and 6 are schematic views showing one example of an open cell stent, and Table 1 lists the details of the respective wire stents. In this case, the open cell stent I as listed in Table 1 corresponds to that shown in FIG. 5, and the open cell stent II as listed in Table 1 corresponds to that shown in FIG. 6. The open cell stent I was manufactured at the same surface area as that of the closed cell stent, and the open cell stent II was manufactured so that the struts have the same width as that in the closed cell stent. The wire stents were manufactured so that all the wire stents have a length of 18 mm and a diameter after swelling of 3.5 mm.

TABLE 1

| | Number of main | Number of connected | | | |
|---|---|---|---|---|---|
| | cells in circum- | cells in circum- | Width of | Surface area | Weight of |
| | ferential direction | ferential direction | strut (μm) | of stent (mm²) | stent (g) |
| Closed cell stent | 9 cells | 9 cells | 120 | 52.75 | 0.0212 ± 0.0002 |
| Open cell stent I | 9 cells | 3 cells | 125 | 52.58 | 0.0212 ± 0.0003 |
| Open cell stent II | 9 cells | 3 cells | 120 | 51.98 | 0.0207 ± 0.0001 |

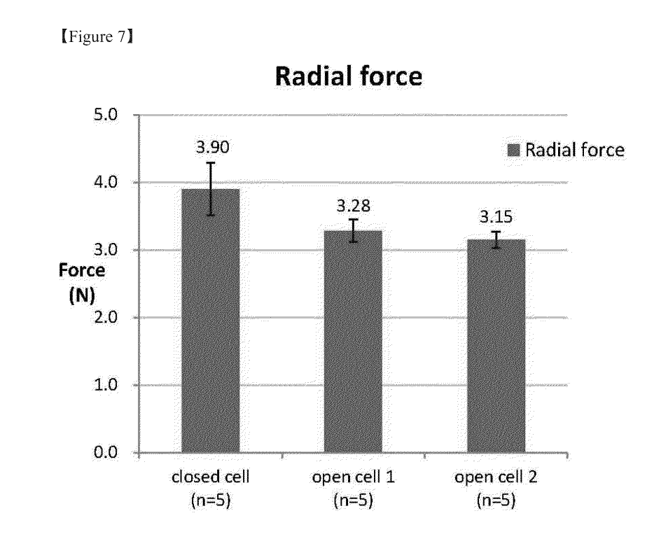

FIG. 7 is a graph illustrating the results obtained by comparing the radial forces of the closed cell stent and the open cell stent. The assay conditions are as follows: a sample size of 3.5×18 mm, a load cell of 250 N, and a rate of 1 mm/min. Here, the closed and open cell stents were pressured to a diameter of 50%, and forces applied to the closed and open cell stents were measured.

As shown in FIG. 7, it was revealed that the radial force of the closed cell stent was approximately 19% higher than the open cell stent I, and approximately 24% higher than the open cell stent II.

Hereinafter, one exemplary embodiment of the wire stent capable of promoting endothelial cellularization according to the present invention will be described in further detail with reference to the accompanying drawings.

FIG. 9 is a configuration view schematically showing the cross section of a strut in the wire stent according to one exemplary embodiment of the present invention. As shown in FIG. 9, struts 10 constituting the wire stent are preferably formed so that the width of each of the struts 10 increases toward the inner wall of a blood vessel (a portion positioned below the strut and indicated by double slashes).

That is, the strut 10 may be formed so that the width of the strut 10 increases, as shown in FIG. 9, in a state in which the strut 10 is mounted at the inner wall of the blood vessel. The strut 10 as described above is configured to allow the endothelial cells to easily climb over the strut 10 in both directions of the strut 10 (in a direction of arrows or a direction opposite to the direction of arrows as shown in the drawing) in a state in which the wire stent is mount at the inner wall of the blood vessel. When the endothelial cells easily climb over the strut 10 in both directions of the strut 10, the endothelial cellularization may be promoted. Therefore, the wire stent may be stably fixed in the inner wall of the blood vessel, which is greatly helpful in treating angiostenosis.

On the other hand, both lateral surface portions of the strut 10 may be formed aslant in a straight or curved line. That is, the both lateral surface portions of the strut 10 may be formed aslant so that the endothelial cells easily climb over the strut 10.

Referring to the shape shown in FIG. 9 among the shapes of such a strut 10, both lateral surfaces of the strut 10 may be formed so that the slope of the strut increases toward the center thereof (the highest portion). When both lateral surfaces of the strut 10 are formed as described above, a portion of the strut toward which the endothelial cells migrate has a low slope, and thus the endothelial cells may migrate toward the portion of the strut. Then, the endothelial cells may easily climb over the strut along a curve surface of the strut having an increasing slope.

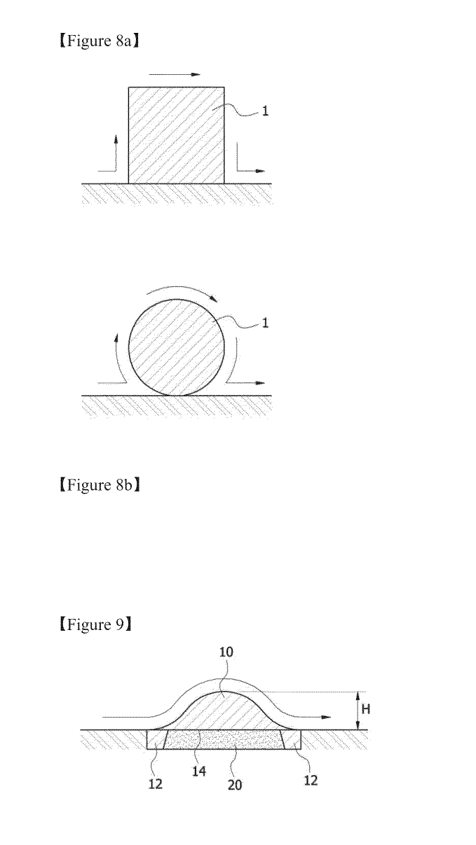

Since the conventional strut as shown in FIG. 8 is formed at a right angle, or formed in a reverse slope in a direction opposite to a traveling direction, the endothelial cells do not easily migrate over the strut.

Also, both lateral surface portions of the strut 10 are preferably formed symmetrically to each other with respect to the center thereof, as shown in FIG. 9. When both lateral surface portions of the strut 10 are formed symmetrically to each other, the endothelial cells may smoothly climb to the highest portion, and then smoothly move down past the highest portion.

Meanwhile, the height H of the strut 10 may be in a range of 30 to 120 μm, particularly preferably less than or equal to 70 μm. In the prior art, the strut 10 was generally designed to have a height of 85 to 90 μm. According to this exemplary embodiment, however, the strut 10 was designed so that the endothelial cells more easily climb over the strut 10 by reducing the height of the strut 10.

Also, a drug-carrying groove 14 configured to carry a drug 20 may be formed on one surface of the strut 10, that is, one surface of the strut 10 which is closely attached to the inner wall of a blood vessel. When the drug-carrying groove 14 is formed as described above, it is possible to deliver a drug capable of suppressing the excessive growth of neointimal cells. For reference, the drug-carrying groove 14 may have protrusions 12 formed on one surface of the strut 10 to extend from both sides thereof.

Next, hereinafter, another exemplary embodiment of the wire stent capable of promoting endothelial cellularization according to the present invention will be described in further detail with reference to the accompanying drawings.

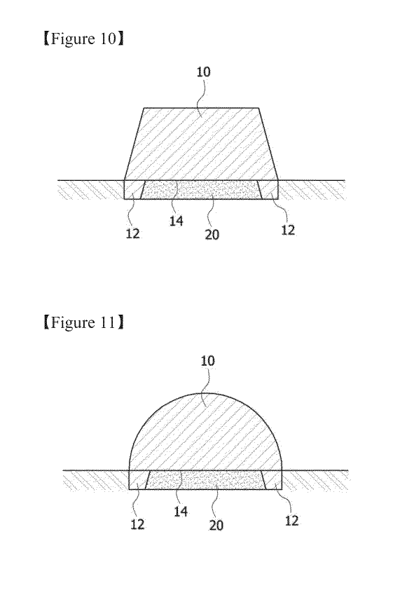

FIGS. 10 and 11 are configuration views schematically showing the cross sections of struts in a wire stent according to another exemplary embodiment of the present invention. For reference, the elements of the wire stent having the same configuration as shown in FIG. 9 have like numbers, and a detailed description thereof is omitted for clarity.

As shown in FIGS. 10 and 11, the strut 10 having a different shape than the exemplary embodiment as shown in FIG. 9 is proposed. According to one exemplary embodiment as shown in FIG. 10, the cross section of the strut 10 is in a trapezoidal shape, and, according to one exemplary embodiment as shown in FIG. 11, the cross section of the strut 10 is in a semicircular shape.

In addition to the exemplary embodiment as shown in FIG. 9, it is evident that the cross section of the strut 10 has various shapes such as a triangular shape, as well as the trapezoidal shape in which both lateral surfaces of the strut 10 are inclined in a straight line as shown in FIG. 10, or the semicircular shape in which both lateral surfaces of the strut 10 are inclined in a curved line as shown in FIG. 11.

As described above, according to the above-described above exemplary embodiments, both lateral surface portions of the strut are configured to promote endothelial cellularization in a state in which the wire stent is mounted at the inner wall of a blood vessel. Therefore, the wire stent may be more firmly fixed in the inner wall of a blood vessel, and thus may be more effective in treating angiostenosis.

Hereinafter, a cell migration assay according to an angle formed between the strut and the inner wall of a blood vessel was performed. Specifically, a total of four types of stents were used. A first stent whose reverse slope is formed at an angle of approximately 30° as shown in FIG. 8b, a second stent whose slope is formed at an angle of approximately 30° as shown in FIG. 9, a third stent whose slope is formed at an angle of approximately 60° as shown in FIG. 10 or 11, and a fourth stent whose slope is formed at an angle of approximately 90° as shown in FIG. 8a were used herein.

Human umbilical vein endothelial cells (HUVEC) were seeded in a 12-well cell culture plate at a density of 2×10⁵ per well, and incubated at 37° C. until the cells adhered to the bottom of the plate.

To form a cell-free zone, a HUVEC monolayer was scraped using a P10 pipette tip.

The cells were washed with a medium to remove the free cells and cell debris.

An embryo germination medium (EGM)-2 medium was added, and each stent was positioned on the cell-free zone.

The cells were incubated for 7 days, and the respective stents were then recovered, and transferred to 1.5 ml tubes.

The stents were washed twice with phosphate buffered saline (PBS), and trypsin-ethylenediaminetetraacetic acid (EDTA) was added at an amount of 0.3 ml (an amount at which the wire stent was immersed). Thereafter, the cells were incubated at 37° C. for 2 minutes to detach the cells from the stents.

The stent were removed, and the cells were centrifuged at 1,000 rpm for 2 minutes to remove 0.25 ml of a supernatant.

A pellet was re-suspended, and the cells were counted.

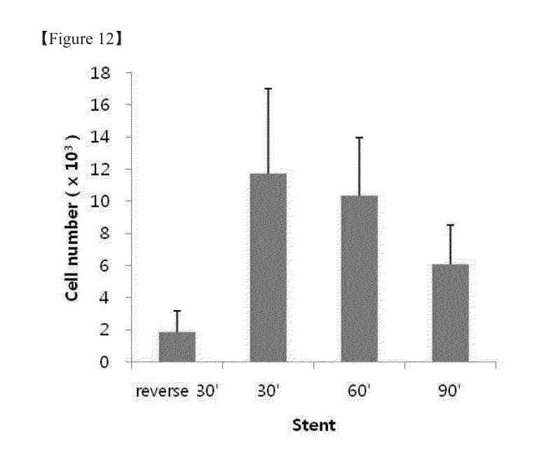

FIG. 12 is a graph comparing the results of a cell migration assay according to an angle formed between the wire strut and the inner wall of a blood vessel. The results showed that the 1.8×10³ cells, the 11.7×10³ cells, the 10.3×10³ cells, and the 6.1×10³ cells were observed on the stent having a reverse slope of 30° and the stents having a slope of 30°, 60°, and 90°, respectively.

As shown in FIG. 12, it was revealed that the cell migration in the stents having a slope of 30° and 60° was approximately twice higher than that in the stent having a slope of 90°, and that the cell migration in the stent having a reverse slope of 30° was approximately three times lower than that in the stent having a reverse slope of 90°.

The present invention has been described in detail. However, it should be understood that the detailed description and specific examples, while indicating preferred embodiments of the invention, are given by way of illustration only, since various changes and modifications within the scope of the invention will become apparent to those skilled in the art from this detailed description.

What is claimed is:

1. A wire stent comprising a plurality of unit wires,
wherein connection portions of the plurality of unit wires come in surface contact with each other in an axial direction.

2. The wire stent of claim 1, wherein the plurality of unit wires are connected to each other using at least one method selected from the group consisting of welding, and adhesion.

3. The wire stent of claim 1, wherein the plurality of unit wires having a predetermined pattern are configured to be connected with each other.

4. The wire stent of claim 1, wherein each of the unit wires comprises a cell wire configured to form a cell with another adjacent unit wire; and a connection unit extending from one end of the cell wire to be connected with another unit wire.

5. The wire stent of claim 4, wherein the cell wire has a zigzag shape in an axial direction.

6. The wire stent of claim 4, wherein connection portions of the connection units come in surface contact with each other.

7. The wire stent of claim 4, wherein the connection units extend in opposite directions from both ends of the cell wire.

8. The wire stent of claim 1, wherein the stent is made of at least one selected from the group consisting of a metal, a biodegradable polymer, a metal coated with the biodegradable polymer, a mixture of the metal and the biodegradable polymer, and a combination thereof.

9. The wire stent of claim 1, wherein, in struts constituting the stent, the struts extending in an axial direction are formed of a biodegradable polymer, and the struts extending in a circumferential direction are formed of a metal.

10. The wire stent of claim 1, wherein the plurality of unit wires are combined to form closed cells.

11. The wire stent of claim 1, wherein each of the struts constituting the stent is formed so that the width of the strut increases toward the inner wall of a blood vessel.

12. The wire stent of claim 11, wherein both lateral surfaces of each of the struts are formed so that a slope of the strut increases toward the center thereof.

13. The wire stent of claim 11, wherein both lateral portions of each of the struts are formed so that the lateral portions of the strut are symmetric to each other.

14. The wire stent of claim 11, wherein a cross section of each of the struts is in a trapezoidal shape.

15. The wire stent of claim 11, wherein the cross section of each of the struts is in a semicircular shape.

16. The wire stent of claim 11, wherein the cross section of each of the struts is in a triangular shape.

17. The wire stent of claim 11, wherein each of the struts has a height of 30 to 120 μm.

18. The wire stent of claim 11, wherein a drug-carrying groove configured to carry a drug is formed at one surface of each of the struts.

19. The wire stent of claim 11, wherein both lateral surface portions of each of the struts constituting the wire stent are formed so that the both lateral surface portions of the strut are inclined in a straight or curved line.

20. A wire stent comprising wires,
wherein each of struts constituting the wire stent is formed so that the width of the strut increases toward the inner wall of a blood vessel.
